(19) United States
(12) Patent Application Publication
(10) Pub. No.: US 2010/0159035 A1
(43) Pub. Date: Jun. 24, 2010

(54) KIT FOR TREATING SKIN INFECTION

(76) Inventor: Avner Shemer, Netanya (IL)

Correspondence Address: BROWDY AND NEIMARK, P.L.L.C.;624 NINTH STREET, NW, SUITE 300, WASHINGTON, DC, 20001-5303, US

(21) Appl. No.: 12/296,280

(22) PCT Filed: Apr. 10, 2007
(86) PCT No.: PCT/IL07/00437

§ 371 Date: Oct. 6, 2008

(30) Foreign Application Priority Data
Apr. 4, 2006 (US) 62219484

(51) Int. Cl. A61K 31/19; A61K 31/60; A61K 31/4164; A61K 36/28; A61K 36/82; A61P 17/00; A61P 31/00; A61P 31/10

(52) U.S. Cl. 424/729; 514/557; 514/163; 514/399; 424/764

(57) ABSTRACT

The invention provides a therapeutic composition, simultaneously containing (1) at least one polar solvent, selected from the group of a short-chain mono-alcohol and a diol; (2) between about 2% and about 25% of at least two keratolytic agents; and (3) a therapeutically safe and effective concentration of a antifungal agent It further provides a kit, consisting of an occlusive device and a therapeutic composition, useful for treatment of fungal skin infection.

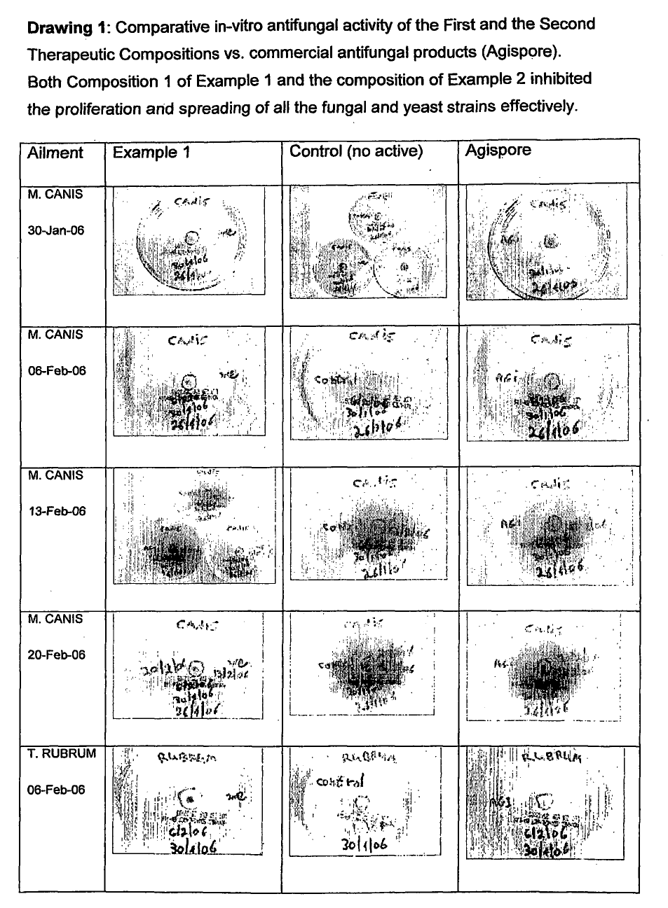

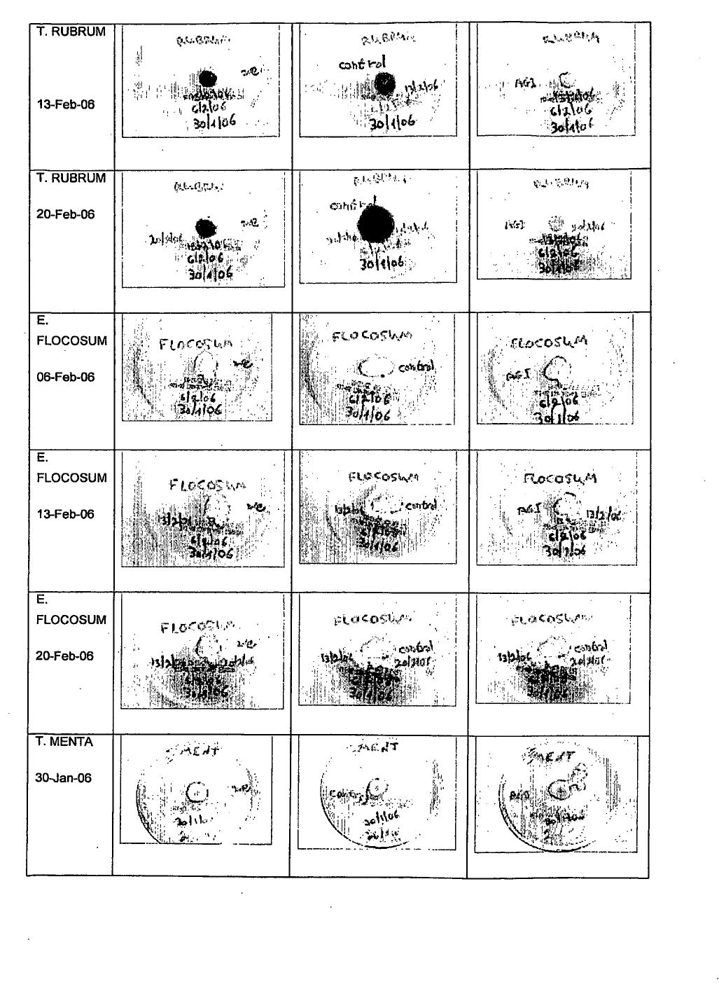

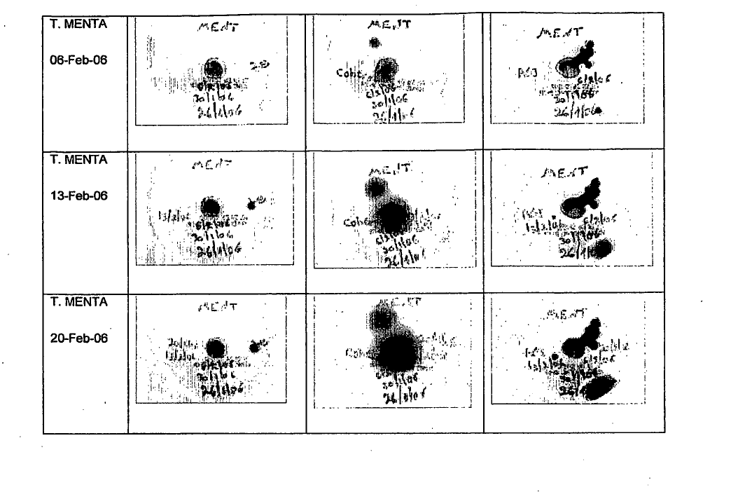

### BACKGROUND

The present invention relates to the treatment of fungal skin infections. In particular, the present invention relates to Dermatophytic infections of the skin.

Dermatophytic infection of the skin can manifest themselves in different anatomical regions of the body and has been accordingly named. Thus, tinea capitis affects the scalp, tinea barbae—the face, tinea unguium—the nails, tinea manuum—the hands, and tinea cruris—the groin area. Tinea pedis, also known as athlete's foot, is a chronic fungal infection of the feet and is the focus of the developments of the present invention. Tinea pedis is estimated to be the second most common skin disease in the United States, behind acne, and up to 15% of the population may manifest the disease.

Tinea pedis presents as pruritic, erythematous, inflamed regions on the feet that may be located on the sole (vesicular type) or lateral aspects (moccasin type) of the foot and sometimes between the toes (interdigital type). Three main genera of fungi may cause tinea pedis, Trichophyton, Epidermophyton, and Microsporum. Other, nondermatophtye, fungi like Malassezia furfur, corynebacterium minutissimum, and Candida species may also cause tinea pedis.

According to the known prior art, for simple cases, Athlete's foot is treated locally with antifungal creams, sprays, liquids and powders based on imidazole antifungals such as clotrimazole and miconazole, as well as zinc undecenoate, allylamines, such as terbinafine, and tolnaflate. Fungal infections usually affect the skin because they live off keratin, a protein that makes up skin, hair and nails.

Prior art data has shown that topical antifungal treatment fails to cure about one-third of patients with tinea pedis (Bell-Syer S E, Hart R, Crawford F, Torgerson D J, Young P, Tyrrell W, Williams H, Russell I: A systematic review of oral treatments for fungal infections of the skin of the feet. J Dermatolog Treat 2001, 12:69-74).

In more severe cases, or if the infection is resistant to usual treatment, antifungal pills may be prescribed. It is important to continue the use of the prescribed antifungal creams and to take all the oral medications properly.

### BRIEF DESCRIPTION OF THE INVENTION

The invention has several aspects. One aspect of the present invention is a first therapeutic composition, comprising (1) at least one polar solvent, selected from the group of a short-chain mono-alcohol and a diol; (2) between about 2% and about 25% of at least two keratolytic agents; and (3) a therapeutically safe and effective concentration of a antifungal agent.

Yet another aspect relates to an occlusive device, intended for retaining a first therapeutic composition at a fungally-infected skin area for an extended period of time.

Another aspect relates to a kit, consisting of an occlusive device and a first therapeutic composition, useful for treatment of fungal skin infection.

A further aspect is a kit comprising an occlusive device and a first therapeutic composition, useful for treatment of said fungal skin infection, and a second therapeutic composition, also useful for treatment of a skin infection.

Still, another aspect is the use of a kit comprising an occlusive device and a first therapeutic composition, with or without the second a therapeutic composition to treat said fungal skin infection.

In further embodiments, a method of treatment of a fungal skin infection, using a kit consisting of an occlusive device and a first therapeutic composition, with or without the second a therapeutic composition.

According to further embodiments of the present invention, the kit is provided in a form selected from the group consisting of a cream, a lotion, a powder or an emulsion.

### BRIEF DESCRIPTION OF THE DRAWINGS

Drawing 1: Comparative in-vitro antifungal activity of the First and the Second Therapeutic Compositions vs. commercial antifungal products. Both Composition 1 of Example 1 and the composition of Example 2 inhibited the proliferation and spreading of all the fungal and yeast strains effectively.

### DETAILED DESCRIPTION OF PREFERRED EMBODIMENTS

### The First Therapeutic Composition

The first therapeutic composition of the present invention includes;
1. between about 2% and about 25% of at least two keratolytic agents;
2. a therapeutically safe and effective concentration of a antifungal agent; and
3. between about 20% and about 80% of at least one polar solvent, selected from the group of a short-chain mono-alcohol and a diol.

Water and optional ingredients are added to complete the total mass to 100%.

All % values are provided herein on a weight (w/w) basis.

### The Keratolytic Agent

The term “keratolytic agent” refers herein to a compound which loosens and removes the stratum corneum of the skin, or alters the structure of the keratin layers of skin.

Suitable keratolytic agents include alpha-hydroxy acids. Non-limiting examples of alpha-hydroxy acids include lactic acid and glycolic acid, malic acid, citric acid and tartaric acid. Alfa hydroxyl acids are keratolytic, and they are also capable of trapping moisture in the skin and initiating the formation of collagen.

Another group of keratolytic agents, suitable for inclusion in the therapeutic composition according to the present invention is beta-hydroxy acids, such as Salicylic acid (o-hydroxybenzoic acid). Beta hydroxyl acids are keratolytic, and they are also have anti-inflammatory and antibacterial properties.

Short chain carboxylic acids (carboxylic acids having up to 6 carbon atoms in their skeleton) are also suitable for inclusion in the therapeutic composition as keratolytic agents. Examples of short chain carboxylic acid include, but are not limited to formic acid, acetic acid, propionic acid, butyric acid (Butanoic acid), valeric acid (pentanoic acid) and caproic acid (hexanoic acid). Also suitable under the definition of short chain carboxylic acid are unsaturated short chain carboxylic acids, i.e., short chain carboxylic acids, having one or more double bonds in their carbon skeleton; and halogenated short chain carboxylic acids, such as fluoroethanoic acid (CH₂FCO₂H), chloroethanoic acid (CH₂ClCO₂H) and dichloroethanoic acid (CHCl₂CO₂H).

In preferred embodiments, the short chain carboxylic acid is selected from the list consisting of formic acid, acetic acid, propionic acid, butyric acid.

Another group of keratolytic agents include phenol and substituted phenolic compounds. Such compounds are known to dissolve and loosen the intracellular matrix of the hyperkeratinized tissue. Dihydroxy benzene and derivatives thereof have been recognized as potent keratolytic agents. Resorcinol (m-dihydroxybenzene) and derivatives thereof are used in anti-acne preparations. Hydroquinone (p-dihydroxybenzene), besides its anti-pigmentation properties, is also keratolytic.

Yet, another class of preferred keratolytic agents includes urea and derivatives thereof. Urea possesses both keratolytic and skin-hydration properties which are beneficial to the damaged tissue of the skin.

In accordance with the present invention, the therapeutic composition includes at least two keratolytic agents. When the at least two or more keratolytic agents are present in the therapeutic composition, a safe and effective peeling agent is attained, which breaks down the keratin layer of the skin, where the microorganisms reside. As a result of such breakedown of the keratin layer, the microorganisms cannot further survive in the infected area.

The combination of at least two keratolytic agents enables a selective breakedown of keratin in infected skin areas, while non-infected skin areas are not affected. This phenomenon is explained by the fact that the keratin layer in fungally-infected skin areas is deformed and thus it is more vulnerable to keratolytic disintegration. Furthermore, combining at least two keratolytic agents facilitates use of each agent in a substantially minimally-irritating concentration, thus decreasing the overall irritation of the therapeutic composition.

In one or more embodiments, the therapeutic composition includes at least two keratolytic agents, from different families of chemicals. Thus, in preferred embodiments of the present invention, the therapeutic composition includes at east two keratolytic agents, from different chemical families, selected from the group consisting of: (1) an alpha-hydroxy acid; (2) a beta-hydroxy acid; (3) a short-chain carboxylic acid; (4) a hydroxyl benzene; and (6) urea. As detailed above, each of these keratolytic agent families may possess, in addition to their keratolytic property, additional therapeutically-beneficial feature, such as anti-inflammatory, skin hydration and antibacterial properties for readily contributing to the overall therapeutic benefit of the therapeutic composition.

In certain embodiments, the therapeutic composition includes at least three keratolytic agents, from different families of chemicals. Thus, in certain embodiments the therapeutic composition contains at least three agents, each from a different chemical family, selected from the group consisting of: (1) a alpha-hydroxy acid; (2) a beta-hydroxy acid; (3) a short-chain carboxylic acid; (4) a hydroxyl benzene; and (6) urea.

### The Antifungal Agent

The first therapeutic composition includes a safe and effective amount of one or more antifungal agents. Preferably, an antifungal agent, is included in the first therapeutic composition of the present invention.

In one or more embodiments, the antifungal agent is an agent that is useful in the treatment, prevention of reducing the severity of a superficial fungal and/or yeast infection of the skin, dermatophytosis, microsporum, trichophyton and epidermophyton infections, candidiasis, oral candidiasis (thrush), candidiasis of the skin, and candida paronychia, which inflicts the nail and nail bed. For the purpose of clarification an a agent that kills fungi and/or yeast is termed “fungicide”.

Thus, in one or more embodiments, the antifungal agent is an azole compound, selected from the group including but not limited to, azoles, diazoles and triazoles, Examples of antifungal azoles include, but are not limited to, azanidazole, bifonazole, butoconazol, chlormidazole, climbazole, cloconazole, clotrimazole, dimetridazole, econazole, enilconazole, fenticonazole, fezatione, fluconazole, flutrimazole, isoconazole, itraconazole, ketoconazole, lanoconazole, metronidazole, metronidazole benzoate, miconazole, neticonazole, nimorazole, niridazole, omoconazol, ornidazole, oxiconazole, posaconazole, propenidazole, ravuconazole, secnidazol, sertaconazole, sulconazole, thiabendazole, tinidazole, tioconazole, voriconazol and salts and derivatives thereof.

In additional embodiments, the antifungal agent is selected from the group consisting of griseofulvin, ciclopirox, ciclopirox-olamine, amorolfine, terbinafine, Amphotericin B, potassium iodide and flucytosine (5FC) at a therapeutically effective concentration.

In a preferred embodiment, the antifungal agent consists of a plant oil or a plant extract possessing antifungal activity; or a plant oil or extract which contains antifungal agents. Non-limiting examples of plants containing agents include, but are not limited to, anise, basil, bergemont, burdock, buchu, chaparral, camphor, cardamom, carrot, canola, cassia, catnip, cedarwood, citronella, clove, couchgrass, cypress, echinacea, eucalyptus, faenia interjecta, c frankincense, garlic, geranium, ginger, grapefruit, holy thistle, hops, hyssop, jasmine, jojova, lavender, lavandin, lemon, lime, mandarin, marigold, marjoram, maytenus ilicifolia, maytenus evonymoides, maytenus aquifolia, micromonospora, myrrh, neroli, nutmeg, orange, ordyceps sinensis, peppermint, perilla, petitgrain, plantain, putterlickia verrucosa, putterlickia pyracantha, putterlickia retrospinosa, rosemary, sage, spearmint, star anise, St. John's wort, red clover, tangerine, tea tree, terfezia clayeryi, thyme vanilla, verbena, white clover and yellow dock.

Yet, in another embodiment, the antifungal agent is an oxidizing agent or a substance that releases free radicals and/or active oxygen. Exemplary oxidizing agents are hydrogen peroxide, benzoyl peroxide, elemental halogen species (compounds), as well as oxygenated halogen species (compounds), bleaching agents (e.g., sodium, calcium or magnesium hypochloride and the like), perchlorite species (compounds), iodine and iodate compounds. Organic oxidizing agents are also included in the definition of “oxidizing agent” according to the present invention, such as quinones. Such agents possess a potent broad spectrum activity

In a particularly preferred embodiment, the antifungal agent is a combination of at least two antifungal agents, as listed hereinabove. Preferably, the antifungal agents in such a combination are selected from different classes of an antifungal agent. For example, a preferred combination of antifungal agents according to the present invention comprises a combination of an imidazole antifungal agent and a plant oil or extract which possesses antifungal activity. By combining antifungal agents from different classes in the therapeutic composition, a synergistic effect is conceivably attained.

The term “safe and effective amount” as used herein, means an amount of an active ingredient high enough to modify the wound condition to be treated or to deliver the desired skin benefit, but low enough to avoid serious side effects, at a reasonable benefit to risk ratio within the scope of sound medical judgment. What is a safe and effective amount of the active ingredient will vary with the specific active, the ability of the active to penetrate through the skin, the age, health condition, and skin condition of the user, and other like factors.

By “pharmaceutically-acceptable salts” are meant any of the commonly-used salts that are suitable for use in contact with the tissues of humans without undue toxicity, irritation, incompatibility, instability, irritation, allergic response, and the like.

In one or more embodiments, the antifungal agent is a combination of at least two antifungal agents, which belong to different families of chemicals, or which are derived from different sources. Thus, in preferred embodiments the antifungal agent contains at east two substances, each belongs to a different chemical family, selected from the list of (1) an azole; (2) a diazole; (3) a triazole; (4) a plant oil or a plant extract which possesses antifungal activity; (5) a plant oil or extract which contains antifungal agents; (6) an oxidizing agent; and (7) a substance that releases free radicals and/or active oxygen. Each of these antifungal agent families act through different antifungal mechanisms, and thus, a combination of at least two antifungal agents conceivably contributes to a synergistic antifungal effect, thereby increasing the therapeutic benefit of the therapeutic composition.

### The Short-Chain Mono-Alcohol and Diol

### Short-Chain Mono-Alcohols, According to the Present Invention

A short-chain alcohol is a compound, having up to 5 carbon atoms in its carbon chain skeleton and one hydroxyl group, such as ethanol, propanol, isopropanol, butanol, iso-butanol, t-butanol and pentanol.

A diol is a compound that contains two hydroxy groups in its molecular structure, such as propylene glycol (e.g., 1,2-propylene glycol and 1,3-propylene glycol), butanediol (e.g., 1,4-butanediol), butanediol (e.g., 1,3-butanediol and 1,4-butenediol), butynediol, pentanediol (e.g., 1,5-pentanediol), hexanediol (e.g., 1,6-hexanediol), octanediol (e.g., 1,8-octanediol), neopentyl glycol, 2-methyl-1,3-propanediol, diethylene glycol, triethylene glycol, tetraethylene glycol, dipropylene glycol and dibutylene glycol.

In one or more embodiment, the polar solvent is a combination of a short-chain alcohol and a diol. The ratio between the short-chain alcohol and the diol can range from about 1:10 and 10:1.

### Additional Components of the Therapeutic Composition

The therapeutic composition of the present invention may further optionally include a variety of formulation excipients, which are added in order to fine-tune the consistency of the formulation, protect the formulation components from degradation and oxidation and modify their consistency. Such excipients may be selected, for example, from emulsifiers, thickening agents, stabilizing agents, antioxidants, humectants, preservatives, colorant and odorant agents and other formulation components, used in the art of formulation.

As shown in laboratory and human experiments, the combination of a keratolytic agent, an antifungal agent, and a polar solvent in a therapeutic composition is sufficient to cause full clearance of an infection after one treatment.

### The Occlusive Device

The occlusive device, according to the present invention, includes a flexible, wearable polymeric body that can conform to the skin surface of a human of mammal subject.

The shape of the occlusive device is designed to conform to the treated organ area. For example, for the treatment of foot infection a boot-shaped polymeric body can be selected. Likewise, in the treatment of an infection of an area of the hand, a glove-shaped polymeric body can be used; and in the treatment of an infected finger or toe, a sleeve-like polymeric body, having suitable dimensions to be wearable onto a finger or toe can be used.

Notwithstanding the above, any polymeric body that can be worn on an affected body and provide an occlusive effect is suitable for use as the occlusive device according to the present invention.

Any flexible polymeric material can be used in constructing the occlusive device according to the present invention. Without derogating from the generality of optional occlusive films, polyethylene, polypropylene, vinyl polymer, latex, rubber, PVA, nitrile polymer and the like.

Optionally, an occlusive bandage can be used to enhance hydration of the infected skin.

Optionally, the composition can include up to 50% of petrolatum to readily facilitate creating an occlusive layer above the infected skin.

### Therapeutic Kit

In one or more embodiments a therapeutic kit is provided. The therapeutic kit includes a first therapeutic composition and an occlusive device. By combining the therapeutic composition and the occlusive device, containing such a therapeutic composition, one attains a highly usable tool for optimal simultaneous keratolytic and antifungal agent effect.

Preferably, the kit is provided in a form selected from the group consisting of a cream, a lotion, a powder or an emulsion

### The Second Therapeutic Composition

The second therapeutic composition of the present invention comprises a semi-solid composition, including (1) between about 20% and about 80% of water; (2) between about 2% and about 25% of at least two keratolytic agents; and (3) a therapeutically safe and effective concentration of an antifungal agent. The definitions of a “keratolytic agent” and a “therapeutically safe and effective concentration of a antifungal antifungal agent” are provided hereinabove in the context of the first therapeutic composition.

As used herein the term “semi-solid composition” shall include, but will not be limited to any composition having viscosity substantially within the range of about 7500 to about 75,000 cps using a Brookfield Viscometer with a ‘C’ spindle with Helipath movement at a spindle speed of 20 rpm and 20-25° C. More preferably, a viscosity between 7500 to about 55,000 cps is suitable.

The second therapeutic composition is intended for daily or intermittent use, following the treatment with the therapeutic kit. Treatment every day or at least twice-weekly, for a period of at least two weeks.

### Method of Treatment

One aspect of the present invention relates to a method of treatment of a skin infection, using either the first therapeutic composition alone, or a kit consisting of an occlusive device and the first therapeutic composition. In one or more embodiments, an effective amount of the first therapeutic composition is directly applied onto the infected area. In one or more embodiments, an effective amount of the first therapeutic composition is poured into the occlusive device, and then the device is attached to the infected area. In specific embodiments, the infected area is inserted into the occlusive device. Without derogating from the generality and versatility of the method of application, the following examples are used to illustrate the method of use:

1. Treatment of tinea pedis, using the first therapeutic composition: an effective amount of the first therapeutic composition is applied to the infected area of the foot.
2. Treatment of tinea pedis, using a kit consisting of an occlusive body and the first therapeutic composition: an effective amount of the first therapeutic composition is added into a boot-shaped polymeric bag, (occlusive device), which constitutes the therapeutic kit. The foot is then inserted into the occlusive device for a period of time sufficient to facilitate a combined keratolytics and antifungal agent effect.
3. Treatment of an infection of an area of the hand, using a kit consisting of an occlusive body and the first therapeutic composition: an effective amount of the first therapeutic composition is added into a glove-shaped polymeric bag (occlusive device), which constitutes the therapeutic kit. The infected hand is then inserted into the occlusive device for a period of time sufficient to facilitate a combined keratolytic and antifungal agent effect.
4. Treatment of a nail infection, using a kit consisting of an occlusive body and the first therapeutic composition: an effective amount of the first therapeutic composition is added into a sleeve-shaped polymeric body (occlusive device), which constitutes the therapeutic kit. The infected hand is then inserted into the occlusive device for a period of time sufficient to facilitate a combined keratolytics and antifungal agent effect.

The application of the therapeutic composition, which includes at least two keratolytic agents, in conjunction with the occlusive device for an extended period of time (such as detailed below) enabled an effective keratolytic effect. Consequently, after the removal of the kit, the effect of the keratolytic agent is noticed by desquamation or peeling of the outer layers of the skin, which occurs during several hours or several days. The antifungal effect of the antifungal agent on keratinophilic fungi, such as Epidermophyton, Trichophyton, and Microsporum species (which grow on keratin) is synergistically enhanced.

The treatment, as described in the above examples can be performed once, or periodically. In many instances, a single treatment can be sufficient, because of the synergistic effect of the components of the first therapeutic composition.

In each treatment, the duration of contact between the infected area and the therapeutic kit is typically between 10 about minutes and several hours. In certain embodiments, the duration is between about 10 minutes and about 2 hours.

Following the treatment with the therapeutic kit, it is useful according to the present invention to proceed in the therapy by applying the second therapeutic composition daily or intermittently every day or at least twice-weekly, for a period of at least two weeks.

### Fields of Use

This invention is useful for topically treating a skin infection which involves an infection by a fungus and/or a yeast.

In one or more embodiments of the present invention, the skin infection is a dermatophytosis (also termed tinea or ringworm). Dermatophytosis is caused by a closely related group of fungi known as dermatophytes which have the ability to utilise keratin as a nutrient source. The dermatophytosis can be found on the scalp, glabrous skin, and nails.

In preferred embodiments of the present invention, the infection contains at least one fungus. In one or more embodiments the fungus is a dermatophite. In one or more embodiments, the dermatophite is selected from the group consisting of epidermophyton floccosum, trichophyton rubrum, trichophyton interdigitale, trichophyton tonsurans, trichophyton violaceum, trichophyton concentricum, trichophyton schoenleinii, trichophyton soudanense, microsporum audouinii, microsporum ferrugineum, trichophyton mentagrophytes, trichophyton equinum, trichophyton erinacei, trichophyton verrucosum, microsporum canis, microsporum gypseum, microsporum nanum and microsporum cookei

In preferred embodiments the skin infection concurrently involves hyperkeratosis (an excessive proliferation of the cells of the cornea), resulting in thickening of the horny layer of the skin. In further preferred embodiments the skin infection concurrently involves hyperkeratosis and an infection by a fungal microorganism.

In a further preferred embodiment, the skin infection is tinea pedis. The tinea pedis can involve and fungal strain, which infects the skin. Non-limiting examples of fungi are nondermatophtye, such as trichophyton, epidermophyton, microsporum. Other exemplary microorganisms that cause skin infection are malassezia furfur, corynebacterium minutissimum, and candida species.

In a certain embodiments, tinea pedis is a hyperkeratotic tinea pedis.

In certain embodiments, the tinea pedis is located on the sole (vesicular type) or lateral aspects (moccasin type) of the foot and sometimes between the toes (interdigital type).

In one or more embodiments, the skin infection is an infection of the skin beneath the female breast which involves candida infection.

It is envisaged that the embodiments of the present invention can be used with skin infections that have a fungal component but is not a classic fungal condition such as dandruff and the like.

In one or more embodiments, the skin infection is an infection of the nail, also termed onychomycosis or tinea unguium.

In one or more embodiments, the skin infection is an infection of the skin beneath a baby's diaper, also commonly termed as diaper rash.

Preferably, the present invention is used together with talcum powder or a substance with similar properties.

Preferably, a pH buffer is used in conjunction thereof to readily prevent degradation of the compositions according to the present invention.

In one or more embodiment, non pharmaceutical additives are added for enhanced effectiveness.

By way of example only, tea tree oil has been found to have beneficial properties when applied to infected skin.

By way of an additional example only, zinc oxide has been found to have beneficial properties when applied to infected skin.

By way of yet an additional example only, cod liver oil has been found to have beneficial properties when applied to infected skin

The terms “treatment” and “therapy” as interchangeably used herein shall include, but will not be limited to any treatment of a fungal skin infection, including: (i) preventing the disease or condition from occurring in a subject which may be predisposed to the disease but has not yet been diagnosed as having it; (ii) inhibiting the disease or condition, i.e. arresting its development; (iii) healing the disease or condition; and (iv) relieving the disease or condition, i.e. causing regression of the disease. In the context of the present invention, relieving the disease, means attaining improvement in the subject condition of a fungal skin infection, including, but not limited to clinical improvement, microbiological improvement and aesthetic improvement.

### EXAMPLES

The device of the present invention may have many shapes and forms. Non-limiting options device configurations are provided in the following examples:

### Example 1

### The First Therapeutic Composition

Formulation No 1

| INGREDIENT | % W/W |
|---|---|
| PROPYLENE GLYCOL | 40.00 |
| ALCOHOL | 20.00 |
| CLIMBAZOLE | 0.20 |
| GLYCOLIC ACID 70% | 12.00 |
| PROPIONIC ACID | 2.00 |
| EXTRAPON MARIGOLD (SYMF) | 4.00 |
| SODIUM HYDROXIDE 20% | 5.00 |
| SALICYLIC ACID | 1.00 |
| WATER | 13.50 |
| TWEEN 20 | 2.00 |
| TEA TREE OIL | 0.30 |

Formulation No 2

| INGREDIENT | % W/W |
|---|---|
| PROPYLENE GLYCOL | 40.00 |
| ALCOHOL | 20.00 |
| CLIMBAZOLE | 0.20 |
| VINIGER | 12.00 |
| PROPIONIC ACID | 2.00 |
| EXTRAPON MARIGOLD (SYMF) | 4.00 |
| SODIUM HYDROXIDE 20% | 0.90 |
| SALICYLIC ACID | 1.00 |
| WATER | 17.50 |
| TWEEN 20 | 2.00 |
| TEA TREE OIL | 0.30 |

### Example 2

### The Second Therapeutic Composition

| INGREDIENT | % W/W |
|---|---|
| PURIFIED WATER | 76.94 |
| GLYCERINE | 5.00 |
| CLIMBAZOLE | 2.00 |
| GLYCOLIC ACID 70% | 2.00 |
| IMIDAZOLIDINYL UREA | 0.30 |
| METHYL PARABEN | 0.20 |
| ZINC PYRITHION 48% | 0.80 |
| SODIUM DODECYL SULFATE | 1.50 |
| SODIUM SULPHITE | 1.00 |
| UREA | 2.00 |
| CETEARYL ALCOHOL | 7.00 |
| TRICLOSAN | 0.15 |
| SILICONE 350 | 0.50 |
| P-HYDROXY BENZOIC ACID | 0.01 |
| GERANIUM OIL | 0.60 |

### Example 3

### Clinical Trial Summary

Patients and Methods: 50 patients with tinea pedis were enrolled. All patients had positive fungal infection of their feet's skin by direct smear and culture.

Patients were treated in two stages. In Stage 1, patient's feet were placed in boots-shaped polyethylene occlusive sleeves, filled with about 50 mL of the first therapeutic composition. The feet remained in the sleeves for a period 45-60 minutes, after which the sleeve was removed. During the next 10 days, skin peeling was noticed, until the feet looked clear of infection. 10 days after treatment, 85% of the patients exhibited negative results of fungal infection by direct smear and culture (complete cure).

In stage 2, the second therapeutic composition was applied once daily for 4 weeks.

Results: Of the 45 patients, who completed the first stage, 38 patients (85%) had complete cure, 2 patients had marked improvement, 3 patients had moderate improvement and 2 patients had mild improvement of their feet's skin condition.

### Example 4

### Comparative In-Vitro Antifungal Activity of the First and the Second Therapeutic Compositions vs. Commercial Antifungal Products

A comparative in-vitro study was set to evaluate the effect of Composition 1 of Example 1 and the composition of Example 2, in comparison with commercially available antifungal products, i.e., Lamisil spray (1% terbinafine) and Agispore Solution (1% bifonazole).

Methods: Three fungal strains (epidermophyton floccosum, trichophyton mentagrophytes and trichophyton rubrum) and one yeast (candida albicans) were seeded in the center of a Petri dish, and then, were surrounded by a film containing each of the compositions, using a swab, soaked with each of the compositions. The proliferation and spreading of the microorganisms was followed up for 14 day by visual and photographic observations.

Results: Both Composition 1 of Example 1 and the composition of Example 2 inhibited the proliferation and spreading of all the fungal and yeast strains effectively.

The details of this study for the composition of Example 1, Formulation No. 1, are shown in the photos laid out in Drawing 1.

1. A therapeutic composition for the treatment of a skin infection comprising:
i) between about 2% and about 25% of at least two keratolytic agent;
ii) a therapeutically safe and effective concentration of a antifungal agent; and
iii) between about 20% and about 80% of at least one polar solvent, selected from the group of a short-chain mono-alcohol and a diol.

2. The composition of claim 1, wherein said at least two keratolytic agents are selected from the group consisting of (1) an alpha-hydroxy acids; (2) a beta-hydroxy acid; (3) a short chain carboxylic acid, having up to 6 carbon atoms in their skeleton; (4) a phenol and a substituted phenolic compounds; and (6) urea.

3. The composition of claim 2, wherein said keratolytic agents are selected from the group consisting of:
i) an alpha-hydroxy acid, selected from the group consisting of lactic acid and glycolic acid, malic acid, citric acid and tartaric acid.
ii) salicylic acid
iii) a short chain carboxylic acid, selected from the group consisting of formic acid, acetic acid, propionic acid, butyric acid (Butanoic acid), valeric acid (pentanoic acid) and caproic acid (hexanoic acid),
iv) an unsaturated short chain carboxylic acid; a halogenated short chain carboxylic acid; and a halogenated short chain carboxylic acid selected from the group consisting of fluoroethanoic acid, chloroethanoic acid and dichloroethanoic acid.
v) A phenol, selected from the group consisting of dihydroxy benzene, resorcinol and hydroquinone.
and derivatives thereof.

4. The composition of claim 4, wherein said therapeutic composition includes at least two keratolytic agents, from different families of chemicals.

5. The composition of claim 4, wherein said therapeutic composition includes at least two agents, each from a different chemical family, selected from the group consisting of: (1) a alpha-hydroxy acid; (2) a beta-hydroxy acid; (3) a short-chain carboxylic acid; (4) a hydroxyl benzene; and (5) urea.

6. The composition of claim 2, wherein said therapeutic composition includes at least three keratolytic agents, from different families of chemicals.

7. The composition of claim 6, wherein said keratolytic agent includes at least three agents, each from a different chemical family, selected from the group consisting of (1) a alpha-hydroxy acid; (2) a beta-hydroxy acid; (3) a short-chain carboxylic acid; (4) a hydroxyl benzene; and (5) urea.

8. The composition of claim 1, wherein said antifungal agent is an agent effective against the growth of a microorganism, selected from the group consisting of a fungus and a yeast

9. The composition of claim 8, wherein said antifungal agent belongs to a family of substances, selected from the group consisting of: (1) an azole; (2) a diazole; (3) a triazole; (4) a plant oil or a plant extract which possesses antifungal activity; (5) a plant oil or extract which contains antifungal agents; (6) an oxidizing agent; and (7) a substance that releases free radicals and/or active oxygen.

10. The composition of claim 8, wherein said antifungal antifungal agent belongs to a family of substances, selected from the group consisting of:
i. an antifungal drug, selected from the group of azanidazole, bifonazole, butoconazol, chlormidazole, climbazole, cloconazole, clotrimazole, dimetridazole, econazole, enilconazole, fenticonazole, fezatione, fluconazole, flutrimazole, isoconazole, itraconazole, ketoconazole, lanoconazole, metronidazole, metronidazole benzoate, miconazole, neticonazole, nimorazole, niridazole, omoconazol, ornidazole, oxiconazole, posaconazole, propenidazole, ravuconazole, secnidazol, sertaconazole, sulconazole, thiabendazole, tinidazole, tioconazole, voriconazol, griseofulvin, ciclopirox, ciclopirox-olamine, amorolfine, terbinafine, Amphotericin B, potassium iodide and flucytosine (5FC) at a therapeutically effective concentration.
ii. oil or an extract, derived from a plant, selected from the group consisting of anise, basil, bergemont, burdock, buchu, chaparral, camphor, cardamom, carrot, canola, cassia, catnip, cedarwood, citronella, clove, couchgrass, cypress, echinacea, eucalyptus, faenia interjecta, c frankincense, garlic, geranium, ginger, grapefruit, holy thistle, hops, hyssop, jasmine, jojova, lavender, lavandin, lemon, lime, mandarin, marigold, marjoram, maytenus ilicifolia, maytenus evonymoides, maytenus aquifolia, micromonospora, myrrh, neroli, nutmeg, orange, ordyceps sinensis, peppermint, perilla, petitgrain, plantain, putterlickia verrucosa, putterlickia pyracantha, putterlickia retrospinosa, rosemary, sage, spearmint, star anise, St. John's wort, red clover, tangerine, tea tree, terfezia claveryi, thyme vanilla, verbena, white clover and yellow dock.
iii. an oxidizing agent or a substance that releases free radicals and/or active oxygen, selected from the group consisting of hydrogen peroxide, benzoyl peroxide, an elemental halogen, an oxygenated halogen compound, a bleaching agent, sodium hypochloride, calcium hypochloride, magnesium hypochloride, a perchlorite compound, iodine, an iodate, an organic oxidizing agent, and a quinine.

11. The composition of claim 1, wherein said antifungal agent includes at least two antifungal agents.

12. The composition of claim 11, wherein said antifungal agent substantially contemporaneously includes at least two antifungal agents, from different families of antifungal agents.

13. The composition of claim 12, wherein said antifungal agent substantially contemporaneously includes at least two agents, each from a different chemical family, selected from the group consisting of (1) an azole; (2) a diazole; (3) a triazole; (4) a plant oil or a plant extract which possesses antifungal activity; (5) a plant oil or extract which contains antifungal agents; and (6) an oxidizing agent.

14. The composition of claim 13, wherein said antifungal antifungal agent substantially contemporaneously includes at least three antifungal antifungal agent, from different families of antifungal agents.

15. The composition of claim 14, wherein said antifungal agent substantially contemporaneously includes at least three antifungal agents, each from a different family of antifungal agents, selected from the group consisting of (1) an azole; (2) a diazole; (3) a triazole; (4) a plant oil or a plant extract which possesses antifungal activity; (5) a plant oil or extract including antifungal agents; (6) an oxidizing agent; and (7) a substance that releases free radicals and/or active oxygen.

16. The composition of claim 1, wherein the concentration of the antifungal agent is in a range selected from (1) about 0.001% to about 20%; (2) about 0.01% to about 10%; and (3) about 0.025% to about 5% by weight of the composition.

17. The composition of claim 1, wherein said short-chain mono-alcohol or diol is selected from the group consisting of ethanol, propanol, isopropanol, butanol, iso-butanol, t-butanol, pentanol, propylene glycol, butanediol, butenediol, butynediol, pentanediol, hexanediol, octanediol, neopentyl glycol, 2-methyl-1,3-propanediol, diethylene glycol, triethylene glycol, tetraethylene glycol, dipropylene glycol and dibutylene glycol.

18. The composition of claim 1, further substantially contemporaneously comprising a short-chain mono-alcohol and a diol

19. The composition of claim 1, wherein the ratio between said short-chain alcohol and said diol is between about 1:10 and 10:1.

20. A therapeutic kit for enhancing the therapeutic activity of the composition of claim 1, consisting of the therapeutic composition of claim 1, and a wearable occlusive device.

21. The kit of claim 20, wherein said occlusive device is a flexible, wearable polymeric body readily conformable to the skin surface of a human or mammal subject

22. The kit of claim 21, wherein said shape of the occlusive device is selected from a boot-shaped polymeric body; a glove-shaped polymeric body; and a sleeve-like polymeric body.

23. The kit of claim 21, wherein said occlusive device is constructed of a polymer selected from the group consisting of a polyethylene, a polypropylene, a vinyl polymer, a latex, a rubber, a PVA and a nitrite polymer.

24. A semi-solid therapeutic composition, comprising (1) between about 20% and about 80% of water; (2) between about 2% and about 25% of at least two keratolytic agents; and (3) a therapeutically safe and effective concentration of an antifungal agent.

25. A Method of treatment of a fungal skin infection, comprising:
i. selection of an infected area for treatment; and
ii. administration of the composition of claim 1 to the infected area

26. A Method of treatment of a skin disorder, involving a fungal infection, by simultaneously exerting a keratolytic effect and an antifungal effect comprising:
i. selection of an infected area for treatment;
ii. selection of a wearable occlusive device, suitable for wearing onto the infected area;
iii. application of a kit consisting of said occlusive device and the first therapeutic composition onto the infected area for a set duration of treatment; and
iv. removing the kit from the infected area

27. The method of claim 26, wherein the treatment is performed once, or periodically; and wherein a single treatment is sufficient to cause clearance of the microorganisms from the infected area,

28. The method of claim 26, wherein the duration of treatment is between about 10 minutes and about 2 hours.

29. The method of claim 26, wherein the skin infection involves an infection by a fungus or a yeast.

30. The method of claim 29, wherein the fungus is a dermatophyte.

31. The method of claim 30, wherein the dermatophite is selected from the group consisting of epidermophyton floccosum, trichophyton rubrum, trichophyton interdigitale, trichophyton tonsurans, trichophyton violaceum, trichophyton concentricum, trichophyton schoenleinii, trichophyton soudanense, microsporum audouinii, microsporum ferrugineum, trichophyton mentagrophytes, trichophyton equinum, trichophyton erinacei, trichophyton verrucosum, microsporum canis, microsporum gypseum, microsporum nanum and microsporum cookie.

32. The method of claim 30, wherein the dermatophite is found on the scalp, glabrous skin, or nails.

33. The method of claim 30, wherein the skin disorder is selected from tinea pedis and onychomycosis.

34. The method of claim 33, wherein the tinea pedis is a hyperkeratotic tinea pedis.

35. The method of claim 33, wherein the tinea pedis is located on an area selected from (1) the sole (vesicular type); (2) the lateral aspects of the foot (moccasin type) and (3) between the toes (interdigital type).
